(19) United States
(12) Patent Application Publication
(10) Pub. No.: US 2007/0282268 A1
(43) Pub. Date: Dec. 6, 2007

(54) Thoracentesis catheter system with self-sealing valve

(76) Inventor: Martin L. Mayse, University City, MO (US)

Correspondence Address: Martin L. Mayse, MD, 7360 Teasdale Avenue, Unversity City, MO, 63130, US

(21) Appl. No.: 11/446,086

(22) Filed: May 31, 2006

(51) Int. Cl. A61M 5/178

(52) U.S. Cl. 604/164.01; 604/164.02; 604/167.03

(57) ABSTRACT

The present invention provides thoracentesis systems composed of an insertion needle, a thoracentesis catheter with self-sealing valve, and a valve opening device which are utilized in the removal of fluid or gases from a pleural cavity. The insertion needle is utilized to insert the thoracentesis catheter into the pleural cavity. The thoracentesis catheter provides automatic closure of the flow path from the pleural cavity by automatic closure of the self-sealing valve upon removal of the insertion needle from the thoracentesis catheter. The self-sealing valve prevents drainage of fluid from the pleural cavity and introduction of air into the pleural cavity when the needle is withdrawn from the thoracentesis catheter and a valve opening device is not in place. A drainage flow path from the pleural cavity is established by insertion of a valve opening device into the self-sealing valve of the thoracentesis catheter, thus opening the self-sealing valve. With the self-sealing valve opened by a valve opening device, fluid or gases can be removed from the pleural cavity.

### BACKGROUND OF THE INVENTION

This invention generally relates to medical devices utilized in invasive medical procedures. More specifically, this invention relates to thoracentesis systems composed of an insertion needle, a thoracentesis catheter with self-sealing valve, and a valve opening device which are utilized in combination for the removal of fluid or gases from a pleural cavity.

A thoracentesis procedure is a medical procedure for removing or draining fluid or gases from a pleural cavity of a patient. The thoracentesis procedure includes insertion of a thoracentesis device into a pleural cavity and creating a flow path from the pleural cavity in order to remove fluids or gases from the pleural cavity.

Typically, a needle is utilized to insert a catheter into the pleural cavity. The needle is withdrawn and the catheter remains in the pleural cavity creating a flow path out of the pleural cavity. Negative pressure is applied to the flow path to remove fluid from the pleural cavity.

While performing a thoracentesis procedure, it is important to prevent the introduction of air into the pleural cavity to prevent lung collapse.

Existing thoracentesis devices have exhibited drawbacks. For example, existing devices create a flow path to the pleural cavity immediately upon withdrawal of the needle. If negative pressure is not present or not maintained on the flow path, air can enter the pleural cavity and a risk of lung collapse exists. Some existing devices have included a manually actuated valve to close the flow path upon removal of the needle. However, if the valve is not manually closed, an open flow path through the open valve allows entry of air into to the pleural cavity and a risk of lung collapse exists. Other existing devices have included a combination of a manually actuated valve in series with an automatically sealing valve. This combination closes the flow path to the pleural cavity immediately upon withdrawal of the needle. The flow path to the pleural cavity is reopened upon actuation of the manual valve. These systems are cumbersome to use and when the manually actuated valve is open and negative pressure is not present on the flow path, air can enter into the pleural cavity and a risk of lung collapse exists.

Therefore, needs exist to improve thoracentesis catheter devices utilized in invasive medical procedures. The present invention overcomes many of the limitations of existing thoracentesis devices to improve thoracentesis devices and systems.

### BRIEF SUMMARY OF THE INVENTION

The present invention provides thoracentesis systems composed of an insertion needle, a thoracentesis catheter with self-sealing valve, and a valve opening device utilized in invasive medical procedures. The insertion needle is utilized to insert the thoracentesis catheter with self-sealing valve into the pleural cavity. The needle is withdrawn and the thoracentesis catheter remains in the pleural cavity creating a potential drainage path out of the pleural cavity. The self-sealing valve automatically closes upon removal of the needle from the thoracentesis catheter. Automatic closing of the self-sealing valve prevents fluid from draining out of the pleural cavity through the thoracentesis catheter and prevents air from entering the pleural cavity through the thoracentesis catheter. A flow path from the pleural cavity is established by insertion of the valve opening device into the self-sealing valve of the thoracentesis catheter, opening the self-sealing valve. Negative pressure can be applied through the valve opening device to the thoracentesis catheter in order to remove fluid or gases from the pleural cavity. When desired drainage of the pleural cavity is complete, removal of the valve opening device from the self-sealing valve automatically closes the self-sealing valve and again prevents further fluid from draining out of the pleural cavity through the thoracentesis catheter and air from entering the pleural cavity through the thoracentesis catheter. Insertion of the valve opening device into the self-sealing valve of the thoracentesis catheter may be repeated as needed.

### BRIEF DESCRIPTION OF THE SEVERAL VIEWS OF THE DRAWINGS

FIG. 1 is an elevational view of an insertion needle with syringe and a thoracentesis catheter with self sealing valve made in accordance with the principles of the present invention.

FIG. 2 is a cross-sectional view of the self-sealing valve of the thoracentesis catheter made in accordance with the principles of the present invention.

FIG. 3 is a partial cross-sectional view of the self-sealing valve of the thoracentesis catheter made in accordance with the principles of the present invention with the insertion needle in place.

FIG. 4 is an elevational view of a valve opening device made in accordance with the principles of the present invention.

FIG. 5 is a partial cross-sectional view of the valve of FIG. 3, with the valve opening device of FIG. 4 inserted therein to open the self-sealing valve.

FIG. 6 is an elevational view of a thoracentesis catheter with self-sealing valve and a valve opening device made in accordance with the principles of the present invention connected to a vacuum bottle to produce negative pressure on the thoracentesis catheter to drain fluid or gases from a pleural cavity.

### DETAILED DESCRIPTION OF THE INVENTION

Although the present invention can be made in many different forms, a preferred embodiment is described in this disclosure and shown in the attached drawings. This disclosure exemplifies the principles of the present invention and does not limit the broad aspects of the invention only to the illustrated embodiments.

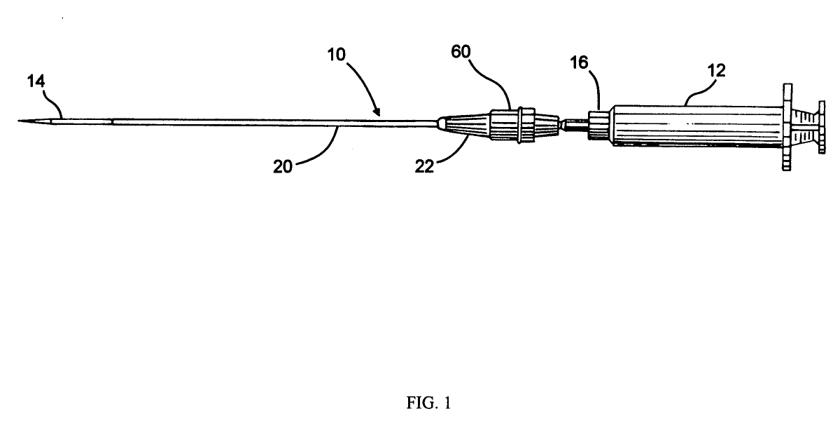

FIG. 1 shows an elevational view of a thoracentesis catheter with self sealing valve 10 made in accordance with the principles of the present invention. The thoracentesis catheter with self sealing valve 10 includes an elongated catheter 20 with a catheter hub 22 and a self-sealing valve 60. The catheter 20 is fixedly attached to the catheter hub 22 and the catheter hub 22 is fixedly attached to the self-sealing valve 60. An elongated insertion needle 14 extends through the catheter 20, through the catheter hub 22 and through the self-sealing valve 60. A syringe 12 is removably connected to the insertion needle 14 by an interference fit or luer fitting 16. The insertion needle 14 is preferably a hollow needle.

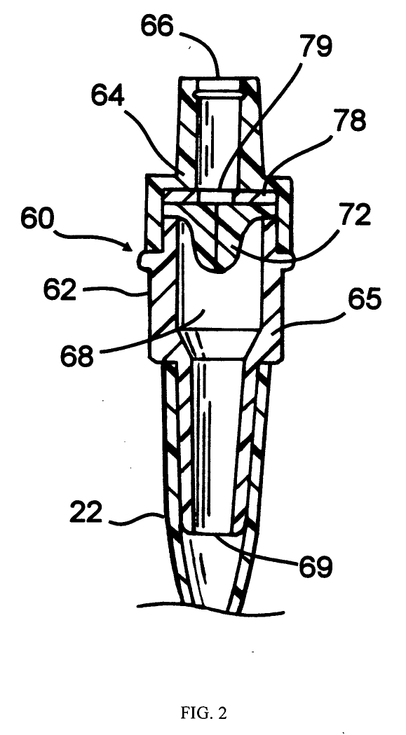

FIG. 2 shows a cross-sectional view of a self-sealing valve 60 made in accordance with the principles of the present invention. FIG. 2 shows a cross-sectional view of the self-sealing valve 60 is shown in a closed position after the insertion needle 14 has been removed from the valve 60. The valve includes a valve body 62 having a proximal portion 64 facing the syringe and a distal portion 65 facing the catheter hub 22. The proximal portion 64 and distal portion 65 of the valve body 62 are fixedly attached to one another. The end 66 of the proximal portion 64 of the valve body 62 and the end 69 of the catheter portion 65 of the valve body 62 each have a hole, and the centers of portions 64 and 65 are hollowed out, thereby forming a passageway 68 through the valve body 62. Positioned within this passageway 68 is a “duckbill” valve 72 which is of the type known in the art consisting of an elastomeric, molded, one-piece dome containing a slit in the center of the dome. The duckbill valve 72 may be opened by inserting an elongated member through the passageway 68 from the end 66 of the proximal portion 64 of the valve body 62 to pry apart the duckbill valve 72. Adjacent to the duckbill valve 72 toward the proximal portion 64 of the valve body 62 is an elastomeric seal 78. The elastomeric seal 78 is a disk-shaped element having a hole 79 through the center to seal against the outside of the insertion needle 14 or valve opening device 110.

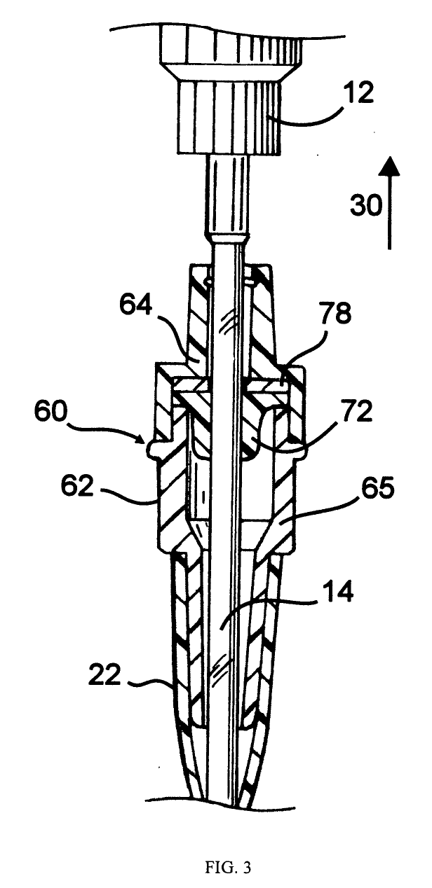

FIG. 3 shows a partial sectional view of the self-sealing valve 60 in an open position, in which a needle 14 extends through the valve 60. The insertion needle 14 can be withdrawn from the self-sealing valve 60 out of the end 66 of the proximal portion 64 of the valve body 62 in the direction of arrow 30. Upon removal of the insertion needle 14 from the self-sealing valve 60, the duckbill valve 72 closes preventing passage of fluid or air through the passageway 68 of the valve body 62.

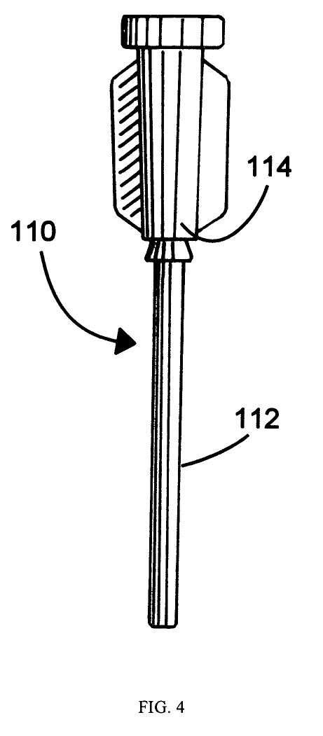

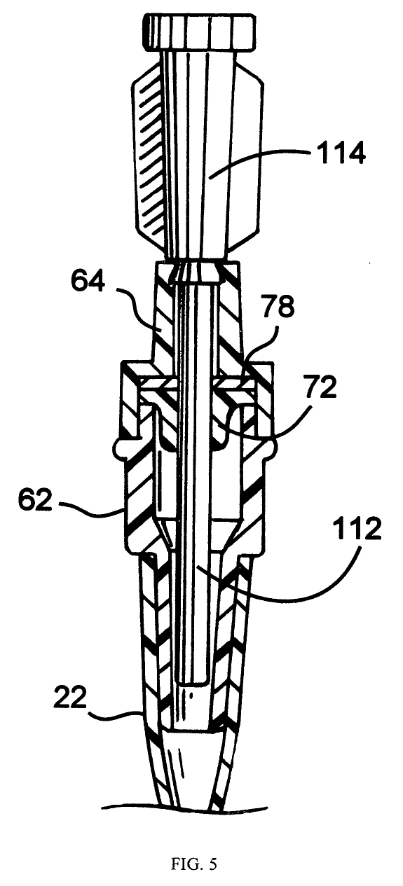

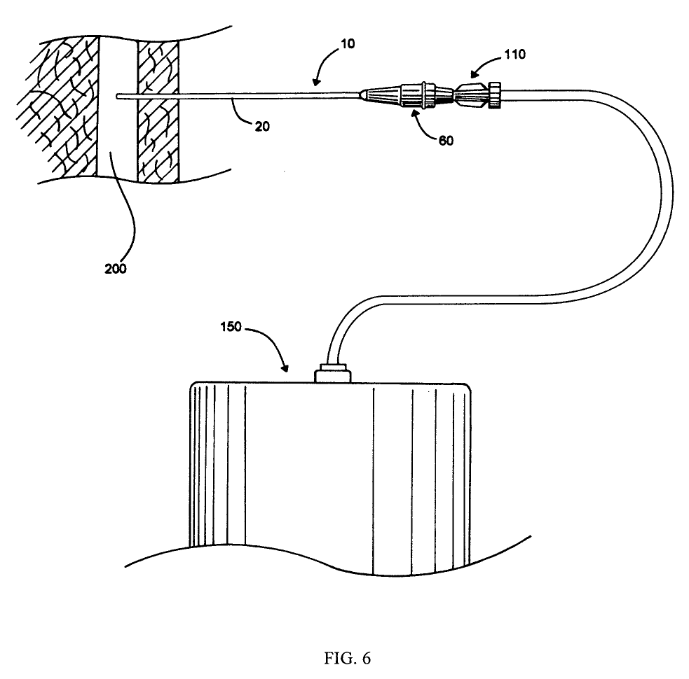

FIG. 4 shows an elevational view of the valve opening device 110 composed of a hollow elongated member 112 fixedly attached to a hollow body 114. The hollow elongated member 112 of the access device may be inserted in to the hollow end 66 of the proximal portion 64 of the body 62 of the self-sealing valve 60 as shown in FIG. 5. The hollow elongated member 112 of the valve opening device 110 is slightly larger in its outside diameter then the hole 79 in the elastomeric seal 78, thereby ensuring that a seal is created between the elastomeric seal 78 and the outside of the hollow elongated member 112 of the valve opening device 110 to prevent fluid from leaking. The insertion of the hollow elongated member 112 of the valve opening device 110 into the hollow end 66 of the proximal portion 64 of the body 62 of the self-sealing valve 60 opens the duckbill valve 72 and thereby allows access to the interior of the catheter 20. The hollow body 114 of the valve opening device 110 may be connected to a negative pressure source, such as a syringe or vacuum bottle 150 as shown in FIG. 6, to draw fluid from the pleural space 200, into and through the catheter 20, the self-sealing valve 60, the valve opening device 110, and into a fluid collection reservoir or vacuum bottle 150. The fluid removal procedure is discontinued by simply withdrawing the hollow elongated member 112 of the access device 110 from the self-sealing valve 60. As the end of the hollow elongated member 112 of the valve opening device 110 comes out of the duckbill valve 72, the valve closes and prevents further fluid from flowing out of the self-sealing valve 60 and also prevents air from entering the catheter 20 and entering into the pleural cavity 200.

What is claimed is:

1. A thoracentesis system comprising;
an elongated hollow flexible catheter having a distal end and a proximal end,
a self-sealing valve means connected to the proximal end of said catheter and in line therewith, said valve means capable of automatically moving from an open position to a closed thereby blocking the passage through the valve means, said valve means capable of moving from a closed position to an open position upon insertion of any valve opening means into said valve means,
a hollow needle having a sharpened distal end adapted to extend through said catheter and valve means and beyond the distal end of said catheter, said needle being operable for complete withdrawl from said catheter and said valve means, upon complete withdrawl of said needle from said valve means the valve moves from an open position to a closed,
a means connected to the proximate end of said hollow needle for applying negative pressure to said needle,
a valve opening means capable of moving said valve means from the a closed position to an open position upon insertion into the valve means,
a means connected to the proximal end of said valve opening means for applying negative pressure to said needle.

2. A self-sealing valve comprising;
a valve body with a proximal end, a distal end, and a passageway through the valve body,
an elastomeric valve movable between an open position and a closed position, when in the open position the passageway through the valve body is open and capable of allowing air and fluid to move freely though the passageway, when in the closed position the passageway through the valve is closed preventing air and fluid from moving through the passageway,

3. The self-sealing valve of claim 2 wherein said valve is adapted to have a needle extend through the elastomeric valve such that the needle keeps the elastomeric valve open.

4. The self-sealing valve of claim 3 wherein the elastomeric valve moves from an open position to a closed position upon complete withdrawl of said needle from said elastomeric valve.

5. The self-sealing valve of claim 4 wherein the elastomeric valve moves from a closed to an open position upon insertion of a valve opening means into said elastomeric valve.

6. A valve opening device comprising;
a hollow elongated member capable of moving the elastomeric valve of the self-sealing valve of claim 2 from a closed position to an open position,

7. The valve opening device of claim 6 with a proximal connecting means capable of connecting to a negative pressure source.
